(19) United States
(12) Patent Application Publication
(10) Pub. No.: US 2025/0177706 A1
(43) Pub. Date: Jun. 5, 2025

(54) ANGIOPLASTY BALLOON CATHETER COMPRISING A VARIABLE DIAMETER SHAFT

(71) Applicant: Timothy Murphy, Cambridge, MA (US)

(72) Inventor: Timothy Murphy, Cambridge, MA (US)

(21) Appl. No.: 19/048,931

(22) Filed: Feb. 9, 2025

Related U.S. Application Data
(63) Continuation-in-part of application No. 16/866,907, filed on May 5, 2020, now abandoned.

(51) Int. Cl. A61M 25/10; A61M 25/00

(52) U.S. Cl. CPC A61M 25/104; A61M 25/0023; A61M 2202/0007; A61M 2202/04; A61M 2210/12

(57) ABSTRACT

The disclosed invention is in the field of medical devices, namely angioplasty balloon catheters. The embodiments provide an angioplasty balloon catheter that has favorable features of a variable diameter shaft, such as pushability, torque, and trackability, enabled by a transition between a proximal shaft of a first diameter and a distal shaft of a second smaller diameter. Other favorable features of the invention include the ability to obturate bleeding when placed in a blood vessel without an enveloping sheath, and the ability of the larger proximal shaft to accommodate fluid injection functions.

### CROSS-REFERENCE TO RELATED APPLICATIONS

This application is a Continuation-In-Part under 35 U.S.C. § 120 and 37 CFR 1.53(b) of both patent applications Ser. No. 16/866,907, EFS ID 39354524, title “Multifunctional Angioplasty and Angiography Catheter Convertible Between Over-the-Wire and Rapid-Exchange Configurations and Methods of Use Thereof,” filed 5, May, 2020, and 18/106,262, EFS ID 47488149, title “Angioplasty Balloon Catheter Comprising A Biaxial-Coaxial Transition,” filed 6, Feb. 2023, both of which are incorporated herein in their entireties, and claims the benefit of the prior applications under 35 U.S.C. § 120.

### FIELD OF THE INVENTION

The present invention relates to medical devices and medical vascular interventional methods. More particularly, the present invention is directed to medical devices used for angioplasty of intravascular stenoses, and methods of use thereof.

### BACKGROUND

| U.S. Pat. | | |
|---|---|---|
| Pat. No. | Issue Date | Patentee |
| U.S. Pat. No. 12,011,556 | Jun. 18, 2024 | Sarradon |
| U.S. Pat. No. 11,291,806 | Apr. 05, 2022 | Sarradon |
| U.S. Pat. No. 10,353,358 | Jul. 30, 2019 | Carmel, et al. |
| U.S. Pat. No. 10,149,962 | Dec. 11, 2018 | Franklin |
| U.S. Pat. No. 9,248,263 | Feb. 02, 2016 | Sarradon |
| U.S. Pat. No. 8,532,749 | Sep. 10, 2013 | Patton |
| U.S. Pat. No. 7,873,404 | Jan. 18, 2011 | Patton |
| U.S. Pat. No. 6,702,781 | Mar. 09, 2004 | Reifart |
| U.S. Pat. No. 6,402,720 | Jun. 11, 2002 | Miller |
| U.S. Pat. No. 6,322,577 | Nov. 27, 2001 | McInnes |
| U.S. Pat. No. 6,007,517 | Dec. 28, 1999 | Anderson |
| U.S. Pat. No. 5,538,510 | Jul. 23, 1996 | Fontirroche |
| U.S. Pat. No. 5,489,271 | Feb. 06, 1996 | Anderson |
| U.S. Pat. No. 5,484,412 | Jan. 16, 1996 | Pierpont |
| U.S. Pat. No. 5,484,411 | Jan. 16, 1996 | Inderbitzen et al. |
| U.S. Pat. No. 5,472,425 | Apr. 22, 1994 | Teirstein |
| U.S. Pat. No. 5,472,425 | Dec. 05, 1995 | Teirstein |
| U.S. Pat. No. 5,470,314 | Nov. 28, 1995 | Walinsky |
| U.S. Pat. No. 5,433,706 | Jul. 18, 1995 | Abiuso |
| U.S. Pat. No. 5,425,714 | Jun. 20, 1995 | Johnson et al. |
| U.S. Pat. No. 5,425,709 | Jun. 20, 1995 | Gambale |
| U.S. Pat. No. 5,411,478 | May 02, 1995 | Stillabower |
| U.S. Pat. No. 5,409,458 | Apr. 25, 1995 | Khairkhahan et al. |
| U.S. Pat. No. 5,403,274 | Apr. 04, 1995 | Cannon |
| U.S. Pat. No. 5,395,353 | Mar. 07, 1995 | Scribner |
| U.S. Pat. No. 5,395,333 | Mar. 07, 1995 | Brill |
| U.S. Pat. No. 5,383,890 | Jan. 24, 1995 | Miraki et al. |
| U.S. Pat. No. 5,383,856 | Jan. 24, 1995 | Bersin |
| U.S. Pat. No. 5,383,853 | Jan. 24, 1995 | Jung |
| U.S. Pat. No. 5,378,237 | Jan. 03, 1995 | Boussignac et al. |
| U.S. Pat. No. 5,370,617 | Dec. 06, 1994 | Sahota |
| U.S. Pat. No. 5,346,505 | Sep. 13, 1994 | Leopold |
| U.S. Pat. No. 5,342,297 | Aug. 30, 1994 | Jang |
| U.S. Pat. No. 5,336,184 | Aug. 09, 1994 | Teirstein |
| U.S. Pat. No. 5,336,184 | Aug. 09, 1994 | Teirstein |
| U.S. Pat. No. 5,328,469 | Jul. 12, 1994 | Coletti |
| U.S. Pat. No. 5,324,269 | Jun. 28, 1994 | Miraki |
| U.S. Pat. No. 5,318,535 | Jun. 07, 1994 | Miraki |
| U.S. Pat. No. 5,308,356 | May 03, 1994 | Blackshear, Jr. et al. |
| U.S. Pat. No. 5,295,995 | Mar. 22, 1994 | Kleiman |
| U.S. Pat. No. 5,295,959 | Mar. 22, 1994 | Gurbel et al. |
| U.S. Pat. No. 5,284,473 | Feb. 08, 1994 | Calabria |
| U.S. Pat. No. 5,267,958 | Dec. 07, 1993 | Buchbinder et al. |
| U.S. Pat. No. 5,232,445 | Aug. 03, 1993 | Bonzel |
| U.S. Pat. No. 5,205,822 | Apr. 27, 1993 | Johnson et al. |
| U.S. Pat. No. 5,180,367 | Jan. 19, 1993 | Kontos et al. |
| U.S. Pat. No. 5,171,222 | Dec. 15, 1992 | Euteneuer et al. |
| U.S. Pat. No. 5,158,540 | Oct. 27, 1992 | Wijay et al. |
| U.S. Pat. No. 5,156,594 | Oct. 20, 1992 | Keith |
| U.S. Pat. No. 5,156,594 | Oct. 20, 1992 | Keith |
| U.S. Pat. No. 5,154,725 | Oct. 13, 1992 | Leopold |
| U.S. Pat. No. 5,147,377 | Sep. 15, 1992 | Sahota |
| U.S. Pat. No. 5,135,535 | Aug. 04, 1992 | Kramer |
| U.S. Pat. No. 5,116,318 | May 26, 1992 | Hillstead |
| U.S. Pat. No. 5,108,370 | Apr. 28, 1992 | Walinsky |
| U.S. Pat. No. 5,102,403 | Apr. 07, 1992 | Alt |
| U.S. Pat. No. 5,087,247 | Feb. 11, 1992 | Horn |
| U.S. Pat. No. 5,078,685 | Jan. 07, 1992 | Colliver |
| U.S. Pat. No. 5,061,273 | Oct. 29, 1991 | Yock |
| U.S. Pat. No. 5,061,273 | Oct. 29, 1991 | Yock |
| U.S. Pat. No. 5,049,131 | Sep. 17, 1991 | Deuss |
| U.S. Pat. No. 5,046,503 | Sep. 10, 1991 | Schneiderman |
| U.S. Pat. No. 5,046,497 | Sep. 10, 1991 | Millar |
| U.S. Pat. No. 5,040,548 | Aug. 20, 1991 | Yock |
| U.S. Pat. No. 5,040,548 | Aug. 20, 1991 | Yock |
| U.S. Pat. No. 4,994,033 | Feb. 19, 1991 | Shockey et al. |
| U.S. Pat. No. 4,988,356 | Jan. 29, 1991 | Crittenden |
| U.S. Pat. No. 4,988,356 | Jan. 29, 1991 | Crittenden et al. |
| U.S. Pat. No. 4,983,167 | Jan. 08, 1991 | Sahota |
| U.S. Pat. No. 4,944,745 | Jul. 31, 1990 | Sogard et al. |
| U.S. Pat. No. 4,909,781 | Mar. 20, 1990 | Husted |
| U.S. Pat. No. 4,909,252 | Mar. 20, 1990 | Goldberger |
| U.S. Pat. No. 4,850,969 | Jul. 25, 1989 | Jackson |
| U.S. Pat. No. 4,842,590 | Jun. 27, 1989 | Tanabe |
| U.S. Pat. No. 4,824,435 | Apr. 25, 1989 | Giesy |
| U.S. Pat. No. 4,819,751 | Apr. 11, 1989 | Shimada et al. |
| U.S. Pat. No. 4,790,315 | Dec. 13, 1988 | Mueller, Jr. et |
| U.S. Pat. No. 4,771,777 | Sep. 20, 1988 | Horzewski |
| U.S. Pat. No. 4,762,129 | Aug. 09, 1988 | Bonzel |
| U.S. Pat. No. 4,762,129 | Aug. 09, 1988 | Bonzel |
| U.S. Pat. No. 4,748,982 | Jun. 07, 1988 | Horzewski |
| U.S. Pat. No. 4,641,654 | Feb. 10, 1987 | Samson |
| U.S. Pat. No. 4,601,713 | Jul. 22, 1986 | Fuqua |
| U.S. Pat. No. 4,581,017 | Apr. 08, 1986 | Sahota |
| U.S. Pat. No. 4,581,017 | April, 1986 | Sahota |
| U.S. Pat. No. 4,323,071 | Apr. 06, 1982 | Simpson |
| U.S. Pat. No. 4,195,637 | Apr. 01, 1980 | Gruntzig |
| U.S. Pat. No. 3,382,872 | May 14, 1968 | Rubin |

### U.S. Patent Application

Patent Appl. No. US 2005/0027249 Publication Date Feb. 3, 2005 Reifart

### Foreign Patent Application

German patent application P 39 34 695.1 Rupprecht

### Publications

Nordenstrom, B. Balloon catheters for percutaneous insertion into the vascular system. Acta Radiol 1962;57:411-416.
Nordenstrom, B. New instruments for catheterization and angiocardiography.Radiology 1965;85:256-259.

This description of art is not intended to constitute an admission that any patent, publication, or other information referred to is “prior art” with respect to the invention unless specifically designated as such. In addition, this section should not be construed to mean that a search has been made or that no other pertinent information as defined in 37 C.F.R. § 1.56(a) exists.

An angioplasty balloon catheter comprises a tubular element further comprising an expansile element generally disposed toward a distal end. Generally, angioplasty balloon catheters comprise at least two lumens, which are tubular channels surrounded by catheter walls, said tubular channels commonly being generally round in cross-section but also comprising semi-circles, crescents, or other shapes in cross-section, each lumen further comprising a longitudinal axis generally disposed along said lumen through a point generally in the center of a cross-section of a substantially circular lumen, or through a point in a cross-section of a noncircular lumen generally located substantially equidistant on average from every point located on a circumference of said noncircular lumen cross-section as possible such that the sum of all radii connecting said center of said longitudinal axis is the smallest possible value, said at least two lumens comprising at least a first lumen for passage of a guide wire or injection of fluids there through, and at least a second lumen as a means for inflation of said expansile member.

Angioplasty balloon catheters typically comprise at least two lumens that can be disposed in relation to each other in either a side-by-side arrangement (“biaxial”), or alternatively a concentric arrangement, said concentric arrangement being substantially a tube-within-a-tube (“coaxial”). Biaxial angioplasty balloon catheters typically have larger outer diameters (O.D.'s) than coaxial angioplasty balloon catheters, and can be extruded together, reducing manufacturing costs compare to coaxial angioplasty balloon catheters, which are typically extruded separately and must be assembled. Furthermore, larger catheters have more “pushability”, or the ability of the catheter to provide more forward force at a distance when advanced by an operator, and “torqueability” or responsiveness at a tip end from a rotational movement by an operator working at a hub end. Coaxial angioplasty balloon catheters offer efficiency because the cross-sectional area of a second lumen for balloon inflation is an outer lumen, and can be larger for a given cross-sectional area compared to a biaxial angioplasty balloon catheter, providing better balloon inflation and deflation performance compared to biaxial angioplasty balloon catheters of the same O.D. Smaller catheters generally have less pushability and torqueability than larger catheters, but have better “trackability”, or the ability to move forward easily in distal, small blood vessels when advanced by an operator by manipulation of a proximal catheter end.

Angioplasty procedures are usually performed using percutaneous access to a blood vessel by the Seldinger technique. The Seldinger technique involves placement of a needle through the skin into a blood vessel, passage of a guide wire through a lumen of the needle into the blood vessel, and removal of the needle and replacement with a plastic diagnostic catheter. Once Seldinger access of a catheter into a blood vessel is achieved, the guide wire and catheter combination are used by an operator to selectively catheterize a blood vessel with a blockage to be treated, arteriograms are obtained, and then an angioplasty balloon catheter is substituted for the diagnostic catheter and positioned so that an expansile balloon disposed toward a tip end of said angioplasty balloon catheter is generally positioned centrally within said blockage. Said angioplasty balloon is then expanded typically by injection of fluids into a lumen in communication with an interior of said angioplasty balloon, thereby relieving the said blockage. Said expansile balloon is then deflated by aspiration of said injected fluids, and then the catheter is removed entirely from the body.

Angioplasty balloon catheters have been used to dilate blockages in blood vessels for over 45 years. The original angioplasty balloon catheter as used by Gruentzig in 1977 was a fixed-wire platform. Subsequently, Simpson and Robert developed a movable guide wire balloon platform, or “over-the-wire” (“OTW”), U.S. Pat. No. 4,323,071. The OTW configuration includes a lumen used to inflate an angioplasty balloon and usually a second lumen used for passage of a guide wire there through. In the OTW configuration, the guide wire exit port is usually at the distal tip of the catheter, and the guide wire entry port is at the hub or proximal end of the catheter.

In 1984 Bonzel introduced the concept of the “rapid exchange” (“RX”), or monorail, configuration, U.S. Pat. No. 4,762,129. With the RX configuration, the guide wire exit port is at the distal tip of the catheter, but the guide wire entry port is substantially distal to the hub or proximal end of the catheter and proximal to an angioplasty balloon.

Although angioplasty balloon catheters have existed for over 45 years and millions of angioplasty procedures are done annually in the U.S., there is a long-felt unmet need of reducing catheter exchanges between diagnostic catheters and therapeutic angioplasty balloon catheters during an angioplasty procedure. Typically, angioplasty balloon catheters are used only for therapeutic purpose of expanding a blockage in a blood vessel. Although angioplasty balloon catheters typically have a guide wire lumen that can be used for injection of fluids, angioplasty balloon catheters typically are not adapted to perform diagnostic functions such as diagnostic angiography, and using an angioplasty balloon catheter for diagnostic angiography by injection of radiopaque fluids through a guide wire lumen would require the guide wire to be absent during the injection of radiopaque fluids, thereby jeopardizing access into a more distal artery that is ensured when a guide wire is in place. Therefore, diagnostic angiography is typically performed with a diagnostic catheter, then the diagnostic catheter removed and replaced with an angioplasty balloon catheter for performance of an angioplasty, then said angioplasty balloon catheter removed and replaced with a diagnostic catheter again to perform a completion angiography.

### SUMMARY

In an exemplary embodiment of the invention, an angioplasty balloon catheter with a variable diameter shaft combines a proximal shaft with a first diameter and a distal shaft with a second diameter that is smaller than said first diameter. Said proximal shaft comprises at least a first lumen for passage of a guide wire and at least a second lumen for inflation of a balloon, and may in some embodiments of the invention further comprise a dedicated third lumen for injection of physician-directed fluids, or in another embodiment may comprise a first lumen that shares functions of guide wire passage and injection of physician-directed fluids. See Patton (U.S. Pat. Nos. 7,873,404B1, 8,532,749B1).

A tubular shaft may further comprise injection exit holes to allow exit of physician direct fluids injected there through and into a blood vessel. Fluids injected could include radiopaque contrast material, vasodilator medication, anticoagulant medication, or thrombolytic medication, for example, among others.

A variable diameter shaft therefore offers potential advantages of facilitating incorporation of a fluid injection function, provides a larger proximal shaft with superior pushability and torqueability while allowing superior trackability by a smaller diameter distal shaft that serves as the leading segment when traversing stenoses or occlusions or when passing through tortuous blood vessels. Another benefit of the invention is that in contrast to conventional angioplasty balloon catheter designs, the balloon is not the largest diameter along the catheter shaft, and therefore a variable diameter angioplasty balloon catheter enables shealthless angioplasty procedures because the proximal shaft has a larger diameter than a collapsed angioplasty balloon and will obturate the blood vessel entry site even after entry of the balloon there through bare without any covering sheath.

One exemplary way to design a single lumen for multiple functions would be to over-size a lumen such that it is of sufficiently larger cross-sectional area than required for its typical guide wire, so that sufficient flow rates can be achieved when fluids are injected into the same lumen even with a guide wire in place there through. This design would require a hub adapter that conforms to a guide wire in a substantially fluid-tight manner forming a choke around said guide wire, but also incorporating an injection port distal to said fluid-tight choke, such as a Touhy-Borst adapter or a hemostasis valve adapter with an injection side port distal to a valve or alternatively a compressible O-ring or disk that deforms to adapt to a fluid-tight configuration around said guide wire when a component of a hub is tightened or otherwise manipulated by an operator. Once injected, fluids flow around said guide and also require an exit port, which may be accommodated by a distal end hole that is sufficiently larger than a typical guide wire to allow contrast to exit from a distal tip end of the angioplasty balloon catheter. Or, as an alternative exemplary embodiment, an injection exit port may comprise at least one or more sideholes through a sidewall of a catheter shaft, either proximal or distal to an expansile balloon but proximal to a catheter distal end hole. Such a design could be accomplished with a catheter having a biaxial or coaxial lumen configuration. In that exemplary embodiment, said injection exit port could be proximal to a guide wire exit port for a catheter used in a “rapid exchange” configuration, or distal to said guide wire exit port.

However, oversizing a single lumen for multiple functions throughout a length of an angioplasty balloon catheter results in an O.D. that may be larger than desired for performing an angioplasty of a small blood vessel, such as for example a blood vessel in a lower leg or foot. For small vessel angioplasty, coaxial angioplasty balloon catheter configurations are preferred, since they can be made with smaller O.D.'s and yet still have similar balloon inflation-deflation performance as biaxial angioplasty configurations with larger O.D's. For a small vessel angioplasty, when fluid injection is desired, a preferred embodiment of the invention is an angioplasty balloon catheter that combines a biaxial segment proximally that incorporates at least a lumen for inflation-deflation of an expansile balloon and at least a multifunction lumen comprising guide wire passage and injectability, with a smaller distal coaxial segment that only accommodates guide wire passage and inflation-deflation of an expansile member. In such an exemplary embodiment, fluid injectability would only be accommodated through the proximal biaxial segment, and therefore at least an injection exit port would need to be disposed along said biaxial proximal catheter segment, or within a transition between a proximal biaxial segment and a distal coaxial segment.

In accordance with one form of the invention, there is a transition in said shaft between said proximal shaft and said distal shaft, said transition comprising a region wherein said first diameter is reduced to said second diameter along a section of its length, and said transition could range from 1 mm to 200 cm or more in length.

In one exemplary embodiment of the invention, said proximal shaft first diameter may range from an outer diameter (O.D.) of substantially between 3 and 6 French, or between 4 and 5 French, or between 4.5 and 5 French, and said distal second diameter has an O.D. of substantially between 2 and 3 French, or between 2.5 and 3 French. Other embodiments of the invention will comprise shaft diameters in the typical range known in the art, for example, between 2 French and 10 French.

A transition from a first diameter to a second smaller diameter may entail thinning of a wall of a lumen along a long axis of said tubular shaft from said proximal segment to said distal segment.

In an exemplary embodiment, said reduction of a first diameter to a second diameter would entail reduction in a shared guide wire lumen and injection lumen such that after the reduction said guidwire lumen would have an inner diameter (I.D.) minimally larger than said guide wire outer diameter, so that it would not accommodate injection of fluids around said guide wire at a clinically useful rate. Conversely, an oversized shared guide wire and injection lumen in a proximal segment of a first diameter would accommodate a clinically useful rate of injection around a guide wire. In a proximal segment of a first diameter, a lumen ID would be substantially greater than an outer diameter of a guide wire, for example more than 1/1000^{th} of an inch greater, or more than 2/1000^{th} of an inch greater. In contrast, a distal segment of a second diameter would have an ID only 1/1000^{th} of an inch larger that said guide wire OD, or not more than 2/1000^{th} of an inch larger.

### BRIEF DESCRIPTION OF THE DRAWINGS

FIG. 1 depicts a preferred embodiment of the invention, in this case an angioplasty balloon catheter with a variable diameter shaft, as seen from a lateral view and comprising an expansile member generally disposed toward a distal end, in this illustration said expansile member is in a first collapsed configuration.

FIG. 2 shows another preferred embodiment of the invention, in this case an angioplasty balloon catheter with a variable diameter shaft, as seen from a lateral view and comprising an expansile member generally disposed toward a distal end, in this illustration said expansile member is in a second expanded configuration.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Devices and related methods are disclosed that generally involve the invention comprising an angioplasty balloon catheter further comprising a variable shaft diameter.

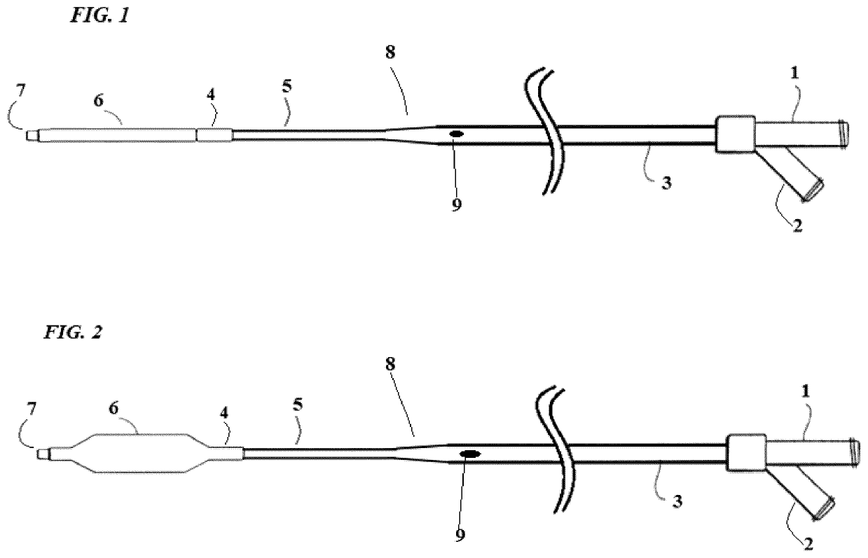

FIG. 1 depicts a preferred embodiment of the invention, in this case an angioplasty balloon catheter with a proximal hub end comprising a first port 1 for passage of a guide wire and injection of fluids there through, said proximal hub end further comprising a second port 2 as a means for injection of fluid or gas as means of inflation of an expansile balloon 6 generally disposed toward a tip end 7 of said angioplasty balloon catheter, said angioplasty balloon catheter further comprising a shaft which is a substantially tubular element generally disposed between said proximal hub end and said tip end 7, said shaft comprising at least two lumens through at least part of its length, including at least a first lumen for passage of a guide wire or injection of fluids there through, and at least a second lumen for injection of fluids or gases as a means of inflation of said expansile balloon 6, said second lumen in fluid communication with an interior of said expansile member 6, in this preferred embodiment said shaft comprising at least a proximal shaft 3 having a first diameter and at least a distal shaft 5 having a second smaller diameter. Said distal shaft 5 further comprises an expansile member, which in this exemplary embodiment has an outer diameter substantially the same as an outer diameter of said distal shaft 5, which is to say an entire distal shaft 5 has a second diameter smaller than a proximal shaft first diameter, including the distal shaft's expansile member in its collapsed configuration. Said angioplasty balloon catheter with variable diameter shaft further comprises in this exemplary embodiment a transition segment 8 wherein said proximal shaft first diameter tapers to a distal shaft second smaller diameter. Said angioplasty balloon catheter in this exemplary embodiment further comprises one or more injection exit holes 9 for exit of fluids injected in this case in a shared guide wire and injection lumen. Those familiar with the art will also recognize that a similar embodiment could comprise three lumens, with one lumen dedicated as a guide wire lumen, a second lumen for balloon inflation and deflation, and a third lumen comprising a dedicated fluid injection lumen, said three-lumen embodiment comprising a hub with three separate ports.

In this FIG. 1 illustration it is shown that in this embodiment an

angioplasty balloon catheter with variable diameter shaft has a proximal shaft 3 comprising a first diameter that is larger than a distal shaft 5 diameter at any point along said distal shaft, including that of the collapsed expansile member 6.

Moreover, in an exemplary embodiment of the invention said expansile member 6 has a proximal cuff 4 that is bonded onto a distal shaft 5. In this embodiment, the distal shaft 5 between the transition segment 8 and the proximal cuff 4 has an outer diameter substantially less that an outer diameter of a proximal shaft 3, for example, greater than a tenth of a millimeter smaller diameter, or between a tenth of a millimeter and half a millimeter, or greater than a half a millimeter up to a millimeter.

Another exemplary embodiment of the invention is an angioplasty balloon catheter with a variable inner diameter, such that a cross sectional area circumscribed by the walls of one or more lumens in a proximal shaft 3 that is in fluid communication with a lumen in a distal shaft 5 is reduced when moving along a long axis from proximal to distal such that a cross sectional area of said one or more lumens is reduced by at least 0.1 mm².

Other embodiments of an angioplasty balloon catheter with variable diameter shaft could comprise more than two diameters and more than one transition.

FIG. 2 is a similar embodiment as shown in FIG. 1 except in this illustration said expansile member 6 is shown inflated. In this exemplary embodiment, it can be appreciated by those skilled in the art that depending on the expansile member expanded diameter, a proximal shaft 3 may be of a first diameter that is larger than a distal shaft 5 second diameter except for said expansile member 6 when said expansile member 6 is inflated, and said expansile member diameter may be larger or smaller than said proximal shaft 3 first diameter, depending on said expansile member 6 expanded diameter. For example, said expansile member 6 may be 1.5 mm diameter when expanded, which could be less than a proximal shaft 3 first diameter but greater than a distal shaft 5 second diameter when excluding the expansile member 6. Alternatively, an expansile member with an expanded diameter of 3 mm could have a larger diameter when inflated than a proximal shaft 3 of 1.7 mm diameter, for example, while still having a smaller second diameter of a distal shaft 5 when said expansile member 6 is in its collapsed configuration.

I claim:

1. An angioplasty balloon catheter comprising a tubular shaft disposed between a hub end and a tip end, said tubular shaft further comprising a variable diameter, said variable diameter comprising at least a proximal shaft with a first outer diameter and at least a distal shaft with a second outer diameter, said second outer diameter smaller than said first outer diameter, said angioplasty balloon catheter further comprising an expansile member disposed toward a distal end of said angioplasty balloon catheter.

2. The angioplasty balloon catheter of claim 1, said tubular shaft further comprising at least two lumens including at least a first lumen for passage of a guidewire or injection of fluids there through and at least a second lumen for injection of fluids or gases as a means to inflate and deflate said expansile balloon.

3. The angioplasty balloon catheter of claim 2, wherein said proximal shaft further comprises a biaxial lumen configuration and said distal shaft further comprising a coaxial lumen configuration.

4. The angioplasty balloon catheter of claim 1, wherein one or more injection exit ports in said tubular shaft are in fluid communication with at least a lumen of said tubular shaft, said one or more injection exit ports further being located along said tubular shaft at a location proximal to said expansile member.

5. The angioplasty balloon catheter of claim 1, wherein said distal shaft second outer diameter is at least 0.1 mm smaller than a first outer diameter at a location between an expansile member proximal cuff and a transition to a proximal shaft.

6. The angioplasty balloon catheter of claim 1, wherein the distal segment second outer diameter over its entire length from tip to distal transition is at least 0.1 mm smaller than a proximal segment first outer diameter over its entire length from hub to proximal transition when said expansile member is in its non-inflated configuration.

7. The angioplasty balloon catheter of claim 1, wherein a transition between said proximal shaft and said distal shaft is located closer to said tip end than to said hub end.

8. The angioplasty balloon catheter of claim 3, wherein said first lumen cross sectional area is variable throughout the length of the tubular shaft.

9. The angioplasty balloon catheter of claim 8, wherein said first lumen cross sectional area in said proximal shaft is at least 0.1 mm² greater than a first lumen cross sectional area in said distal shaft.

10. A method of performing an angioplasty of a blood vessel of a human patient comprising in combination:
a) Gaining percutaneous access to a vascular system in a human patient;
b) Manipulating a guide wire and an angioplasty balloon catheter comprising a tubular shaft disposed between a hub end and a tip end, said angioplasty balloon catheter further comprising an expansile member mounted on said tubular shaft disposed toward said tip end, said tubular shaft comprising at least two lumens including a first lumen for passage of a guidewire or injection of fluids therethrough and at least a second lumen for injection of fluids or gases as a means to inflate and deflate said expansile member, said tubular shaft further comprising a variable diameter, said variable diameter comprising at least a proximal shaft with a first diameter and at least a distal shaft with a second diameter at least 0.1 mm smaller than said first diameter;
c) Injecting fluids or gases into a lumen in communication with an interior of an expansile member generally disposed toward a tip end of said angioplasty balloon catheter to inflate said expansile balloon;
d) Aspirating said fluids or gases to deflate said expansile balloon.

11. The method of performing an angioplasty of a blood vessel of a human patient of claim 10 further comprising injection of physician-directed fluids in a lumen of said angioplasty balloon catheter, said physcian-direct fluids exiting said tubular shaft proximal to said expansile member.
